(19) United States
(12) Patent Application Publication
(10) Pub. No.: US 2010/0137421 A1
(43) Pub. Date: Jun. 3, 2010

(54) SMALL MOLECULE THERAPEUTICS, SYNTHESIS OF ANALOGUES AND DERIVATIVES AND METHODS OF USE

(76) Inventors: Emmanuel Theodorakis, San Diego, CA (US); Ayse Batova, San Diego, CA (US)

Correspondence Address: JONES DAY, 222 EAST 41ST ST, NEW YORK, NY, 10017, US

(21) Appl. No.: 12/514,302

(22) PCT Filed: Nov. 8, 2007
(86) PCT No.: PCT/US2007/023751

§ 371 Date: Feb. 4, 2010

Related U.S. Application Data
(60) Provisional application No. 60/857,997, filed on Nov. 8, 2006.

(51) Int. Cl. A61K 31/352; C07D 311/82; A61P 35/00; A61P 35/02

(52) U.S. Cl. 514/453; 549/224

(57) ABSTRACT

Provided herein are compounds that are inducers of apoptosis activators of caspases and pharmaceutically acceptable derivatives thereof. Also provided are methods of synthesis of the compounds and methods for treatment of diseases in which there is uncontrolled cell growth and spread of abnormal cells, such as cancers, by administering the compounds.

### RELATED APPLICATION DATA

This application claims priority to U.S. provisional application Ser. No. 60/857,997, entitled “Small Molecule Therapeutics, Syntheses Of Analogues And Derivatives And Methods Of Use” to Theodorakis et al., filed Nov. 8, 2006. The contents of the provisional application are incorporated by reference herein in their entirety.

### GRANT INFORMATION

This invention was made with government support under Grant No. CA086079 awarded by National Institute Health. The United States government has certain rights in this invention.

### RELATED APPLICATION DATA

Provided herein are compounds that have activity as inducers of apoptosis and activators of caspases. Also provided are pharmaceutical compositions containing the compounds and methods of using the compounds and compositions.

### BACKGROUND

Tumorigenesis is a multistep process based on genetic alterations that drive the progressive transformation of normal human cells into highly malignant derivatives. It has been suggested that the large diversity of human cancer cell genotypes is a manifestation of six essential alterations in cell physiology that collectively dictate malignant growth (Hanahan, D.; Weinberg, R. A. “The Hallmarks of Cancer” Cell 2000, 100, 57-70). These alterations, also referred to as the hallmarks of cancer, involve: (a) self-sufficiency in growth signals; (b) insensitivity to growth inhibitory signals; (c) evasion of apoptosis; (d) limitless replicating ability; (e) sustained angiogenesis; and (f) tissue invasion and metastasis. These physiologic changes are shared by most, if not all, types of human cancer and represent points for therapeutic intervention. The third alteration from the above list refers to the ability of cancer cells to overcome regular pathways leading to their programmed death (apoptosis) thus expanding their population in a non-controlled manner (MacFarlane, M.; Williams, A. C. “Apoptosis and disease: a life or death decision” EMBO Reports 2004, 5, 674-678).

In general, the apoptotic circuitry is present in latent form in virtually all cell types throughout the body (Lowe, S. W.; Lin, A. W. “Apoptosis in cancer” Carcinogensis 2000, 21, 485-495). Once triggered this program leads to cell deletion via a process that includes chromatin condensation, nuclear fragmentation, cell shrinkage, plasma membrane blebbing and other ultrastructural changes. These changes lead ultimately to phagocytosis by the neighboring cells without inciting inflammatory reactions or tissue scaring.

It has been shown that effectively all traditional anticancer drugs use apoptosis pathways to exert their cytotoxic actions (Reed, J. C. “Dysregulaton of Apoptosis in Cancer”J. Clin. Oncol. 1999, 17, 2941-2953). Since clinically used compounds have some degree of selectivity for cancer cells versus normal cells, screening for apoptosis is a compelling alternative to target-based screening (Lovborg, H.; Gullbo, J.; Larsson, R. “Screening for apoptosis-classical and emerging techniques” Anti-Cancer Drugs 2005, 16, 593-599). Moreover, since the unregulated proliferation of cells can also lead to certain autoimmune and degenerative diseases, therapeutic treatment for these diseases could be accomplished by accelerating the apoptotic process through the administration of small molecules that induce apoptosis.

The tropical trees of the genus Garcinia, found in lowland rainforests of Southeast Asia, are widely known for their use in folk medicines (Kumar, P.; Baslas, R. K. “Phytochemical and the biological studies of the plants of the genus Garcinia” Herba Hungarica 1980, 19, 81-91. Mabberley, D. J. In The plant-book: A portable dictionary of the vascular plants, 2nd ed.; Cambridge University Press: New York, N.Y., 1997, p 293). Phytochemically, these trees are recognized as a rich source of xanthone and xanthonoid natural products that are considered to hold high pharmaceutical potential. (Morton, J. F. In Fruits of warm climates; Dowling, C. F., Jr., Ed.; Julia F. Morton: Miami, Fla., 1987, pp 301-304. Matsumoto, K.; Akao, Y.; Kobayashi, E.; Ohbuchi, K.; Ito, T.; Tanaka, T.; linuma, M.; Nozawa, Y. “Induction of apoptosis by xanthones from mangosteen in human leukemia cell lines” J. Nat. Prod. 2003, 66, 1124-1127. Nakatami, K.; Nakahata, N.; Arakawa, T.; Yasuda, H.; Ohizumi, Y. “Inhibition of cyclooxygenase and prostanglandin E2 synthesis by γ-mansostin, a xanthone derivative in mangosteen, in C6 rat glioma cells. Biochem. Pharmacol. 2002, 63, 73-79. Ho, C.-K.; Huang, Y.-L.; Chen, C.-C. “Garcinone E, a xanthone derivative, has potential cytotoxic effect against hepatocellular carcinoma cell lines” Planta Med. 2002, 68, 975-979.) In fact, a mangosteen-based drink is currently sold to consumers for its tonic, immunostimulant and antioxidant properties. On the other hand, gamboge, the commercially available exudate of Garcinia hanburyi, has been used in traditional Asian medicine for the treatment of indigestion, inflammation and ulcers (Gruenwald, J.; Brendler, T.; and Jaenicke, C. Eds. PDR for Herbal Medicines, 2^{nd} Ed, Medical Economics Co. Montvale, N.J., 2000, pp 325-326).

Several Garcinia natural products exhibit interesting bioactivities and have a documented value in traditional Eastern medicine. For example, morellin (1) was identified as one of the main bioactive metabolites of Garcinia hanburyi and found to exhibit good cytotoxicity against HEL (Human Embryonic Lung fibroplasts) and (HeLa) Henrietta Lacks cervical cancer cells.

Provided herein are small molecules that have similar biological properties with the natural products described above and chemical synthesis process to make the compounds and methods for using the compounds.

### SUMMARY

Provide herein are compounds that are useful in inducing apoptosis. The compounds are of Formula I or Formula II:

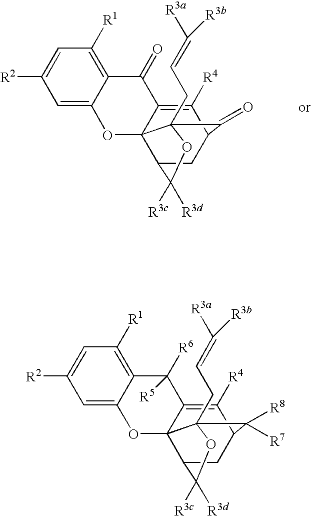

or pharmaceutically acceptable derivatives thereof, wherein the variables are chosen such that the resulting compounds have activity as activators of caspases and inducers of apoptosis.

Pharmaceutical compositions containing a compound of Formula I or II and a pharmaceutically acceptable carrier are provided herein. Also provided are methods for treatment of a variety of clinical conditions in which there is uncontrolled cell growth and spread of abnormal cells, such as in the case of cancer, by administering the compounds and compositions provided herein.

In other embodiments, provided are methods of activating caspases and inducing apoptosis in drug resistant cancer cells, such as breast and prostate cancer cells. In certain embodiments, the compounds provided herein kill the drug resistant cancer cells.

### BRIEF DESCRIPTION OF DRAWINGS

FIG. 1 provides chemical structures of representative compounds (compounds 1-7) from the plant Garcinia morella.

FIG. 2 depicts induction of HUVE cell apoptosis (upper curve) and necrosis (lower curve) by the compound of formula VI at different concentrations after 10 h incubation time; A=A₄₀₅ₙₘ−A₄₉₀ₙₘ; c=concentration of the compound of formula VI.

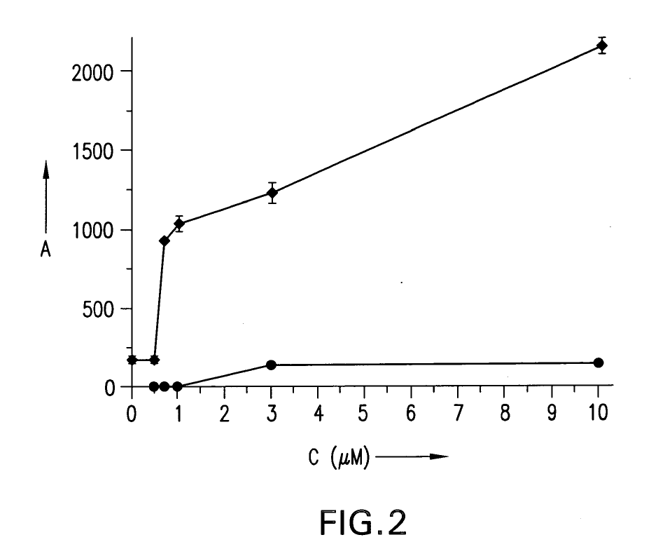

FIG. 3 depicts mean body weight of mice injected with gambogic acid and compound of formula VI relative to control.

### DETAILED DESCRIPTION OF EMBODIMENTS

### A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein “subject” is an animal, such a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, an anti-cancer agent (used interchangeably with “anti-tumor or anti-neoplasm agent”) refers to any agents used in the treatment of cancer. These include any agents, when used alone or in combination with other compounds, that can alleviate, reduce, ameliorate, prevent, or place or maintain in a state of remission of clinical symptoms or diagnostic markers associated with neoplasm, tumor or cancer, and can be used in methods, combinations and compositions provided herein. Non-limiting examples of anti-neoplasm agents include anti-angiogenic agents, alkylating agents, antimetabolite, certain natural products that are anti-neoplasm agents, platinum coordination complexes, anthracenediones, substituted ureas, methylhydrazine derivatives, adrenocortical suppressants, certain hormones, antagonists and anti-cancer polysaccharides.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating a cancer.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, the IC₅₀ refers to an amount, concentration or dosage of a particular test compound that achieves a 50% inhibition of a maximal response in an assay that measures such response.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethene, propene, butene, pentene, acetylene and hexyne. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “heteroalkyl” refers to a straight, branched or cyclic, in certain embodiments straight or branched, aliphatic hydrocarbon group having, inserted in the hydrocarbon chain one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl, in one embodiment having from 1 to about 20 atoms, in another embodiment having from 1 to 12 atoms in the chain.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein, generally selected from Q¹.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, generally selected from Q¹.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

Where the number of any given substituent is not specified (e.g., “haloalkyl”), there may be one or more substituents present. For example, “haloalkyl” may include one or more, in certain embodiments, one, two, three or four of the same or different halogens. As another example, “C₁₋₃alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

Provided herein are compounds that are activators of caspases and inducers of apoptosis. In certain embodiments, compounds provided herein have formula I or II:

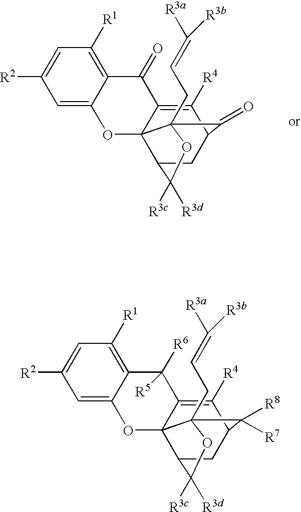

or a pharmaceutically acceptable derivative thereof, wherein

R¹ and R² are each independently hydrogen, alkyl, or —OR^{a};

R^{a} is hydrogen, alkyl, alkenyl, alkynyl, cycloalkyl or aryl;

R^{3a}, R^{3b}, R^{3c} and R^{3d} are each independently hydrogen, alkyl, alkenyl or alkynyl;

R⁴ is hydrogen, halo, pseudohalo, hydroxy, oxo, alkyl, alkenyl, alkynyl, cycloalkyl, aryl, —OR^{a}, —S(O)nR^{b}, —OC(O)R^{b}, —OC(O)OR^{b}, or —NR^{c}R^{d}, —NR^{c}COR^{d} or —NR^{c}SO₂R^{d};

R^{a}, R^{b}, R^{c} and R^{d} are each independently hydrogen, alkyl, alkenyl, alkynyl, aryl, alkylaryl, heterocycle, aralkyl, aralkoxy, cycloalkyl, cycloalkenyl or cycloalkynyl;

n is 0-2;

R⁵ and R⁷ are each independently hydrogen, alkyl, alkenyl or alkynyl; and

R⁶ and R⁸ are each independently hydrogen, alkyl, alkenyl, alkynyl or —OR^{a}.

In one embodiment, R¹ is hydrogen or —OR^{a}. In another embodiment, R¹ is hydrogen or —OH. In one embodiment, R¹ is hydrogen. In one embodiment, R¹ is —OH.

In one embodiment, R² is hydrogen or —OR^{a}. In another embodiment, R² is hydrogen or —OH. In one embodiment, R² is hydrogen. In one embodiment, R² is —OH.

In one embodiment, R^{3a} is hydrogen or lower alkyl. In other embodiment, R^{3a} is hydrogen or methyl. In one embodiment, R^{3a} is hydrogen. In one embodiment, R^{3a} is lower alkyl. In other embodiment, R^{3a} is methyl.

In one embodiment, R^{3b} is hydrogen or lower alkyl. In other embodiment, R^{3b} is hydrogen or methyl. In one embodiment, R^{3b} is hydrogen. In one embodiment, R^{3b} is lower alkyl. In other embodiment, R^{3b} is methyl.

In one embodiment, R^{3c} is hydrogen or lower alkyl. In other embodiment, R^{3c} is hydrogen or methyl. In one embodiment, R^{3c} is hydrogen. In one embodiment, R^{3c} is lower alkyl. In other embodiment, R^{3c} is methyl.

In one embodiment, R^{3d} is hydrogen or lower alkyl. In other embodiment, R^{3d} is hydrogen or methyl. In one embodiment, R^{3d} is hydrogen. In one embodiment, R^{ad} is lower alkyl. In other embodiment, R^{3d} is methyl.

In one embodiment, R^{3a}, R^{3b}, R^{3c} and R^{3d} are each lower alkyl. In one embodiment, R^{3a}, R^{3b}, R^{3c} and R^{3d} are each methyl.

In one embodiment, R⁴ is hydrogen, alkyl or —OR^{a}. In one embodiment, R⁴ is hydrogen or —OH. In one embodiment, R⁴ is hydrogen. In one embodiment, R⁴ is —OH.

In one embodiment, R⁵ and R⁷ are each independently hydrogen or lower alkyl. In one embodiment, R⁵ and R⁷ are each hydrogen.

In one embodiment, R⁶ and R⁸ are each independently hydrogen or —OR^{a}. In one embodiment, R⁶ and R⁸ are each independently hydrogen or —OH. In one embodiment, R⁶ and R⁸ are each hydrogen. In one embodiment, R⁵, R⁶, R⁷ and R⁸ are each hydrogen.

In certain embodiments, R¹, R², R^{3a}, R^{3b}, R^{3c}, R^{3d}, R⁴, R⁵, R⁶, R⁷, R^{a}, R^{b}, R^{c} and R^{d} are each independently optionally substituted with 1, 2, 3 or 4 substituents, each independently selected from Q¹, where Q¹ is halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, heteroalkyl, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, p(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and each Q¹ is independently unsubstituted or substituted with one, two or three substituents, each independently selected from Q²;

each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, heteroalkyl, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;

R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;

R⁵¹, R⁵² and R⁵³ are each independently hydrogen, alkyl, aryl, aralkyl, heteroaryl, heteroaralkyl, heterocyclyl or heterocyclylalkyl;

R⁶⁰ is hydrogen, alkyl, aryl, aralkyl, heteroaryl, heteroaralkyl, heterocyclyl or heterocyclylalkyl; and

R⁶³ is alkoxy, aralkoxy, alkyl, heteroaryl, heterocyclyl, aryl or —NR⁷⁰R⁷¹.

In certain embodiments, Q¹ is halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl, alkynyl, heteroalkyl, cycloalkyl, cycloalkylalkyl, heterocyclyl, heterocyclylalkyl, aryl, heteroaryl, aralkyl, aralkenyl, aralkynyl or heteroarylalkyl. In certain embodiments, Q¹ is alkyl, halo, hydroxy or haloalkyl.

In certain embodiments, the compound has formula III, IV or V:

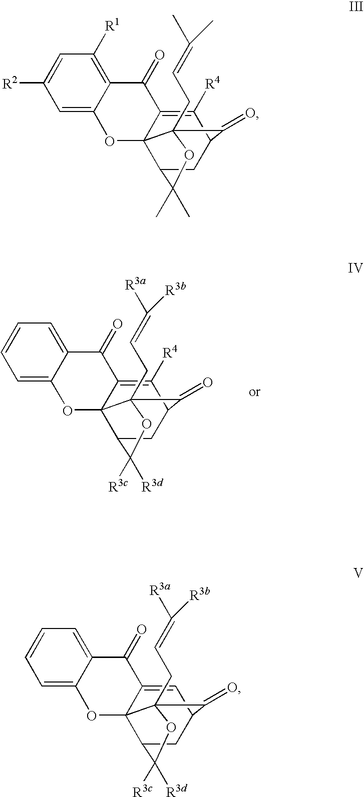

or a pharmaceutically acceptable salt thereof, wherein the variables are as defined elsewhere herein.

In one embodiment, the compound is of formula VI:

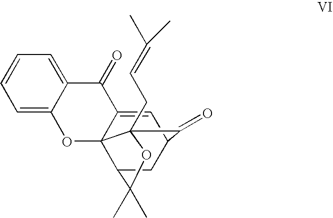

or a pharmaceutically acceptable salt thereof.

### C. Preparation of Compounds

The compounds provided herein can be prepared by methods known to one of skill in the art and following procedures similar to those described in the Examples section herein and routine modifications thereof.

Certain exemplary reaction schemes for the preparation of compounds are illustrated below:

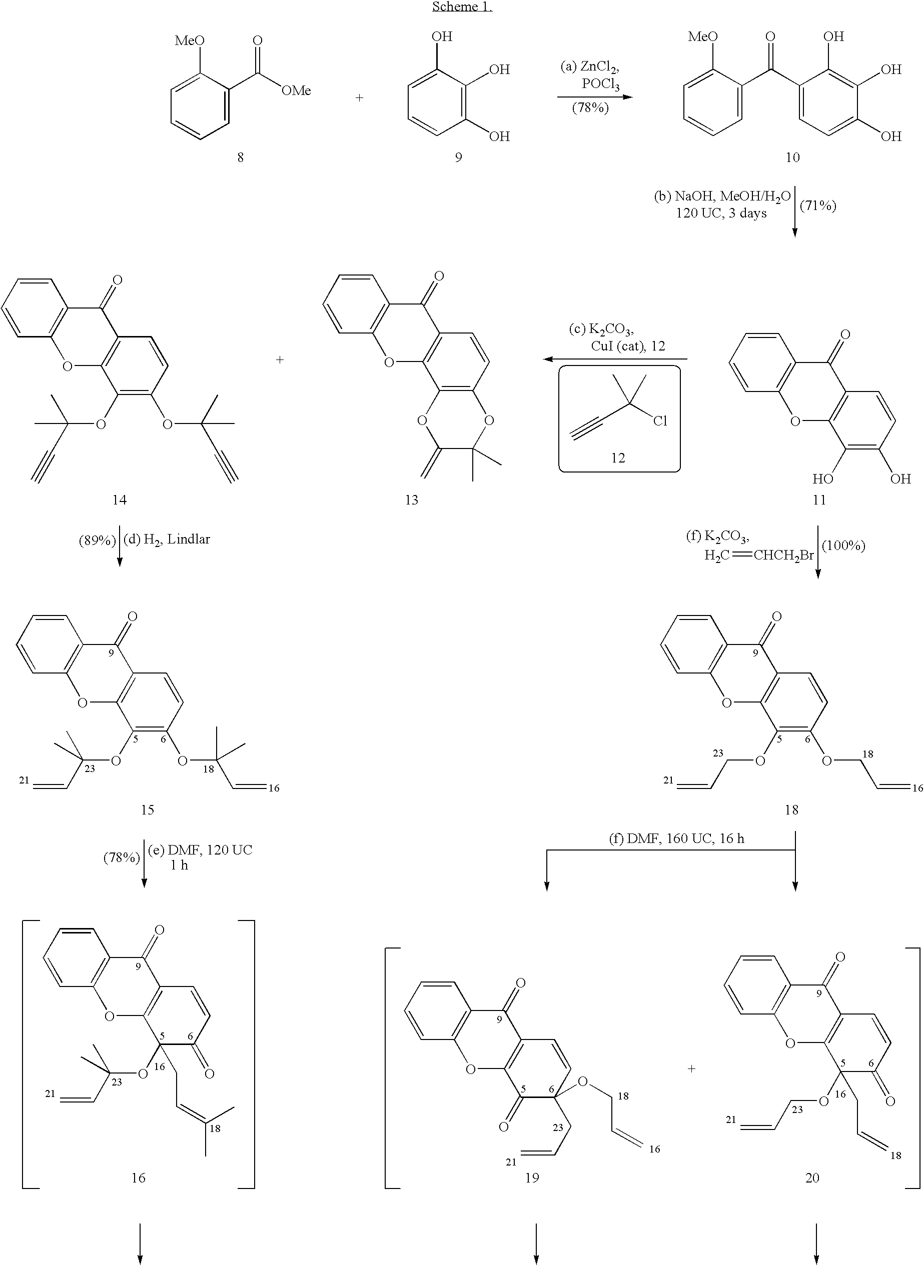

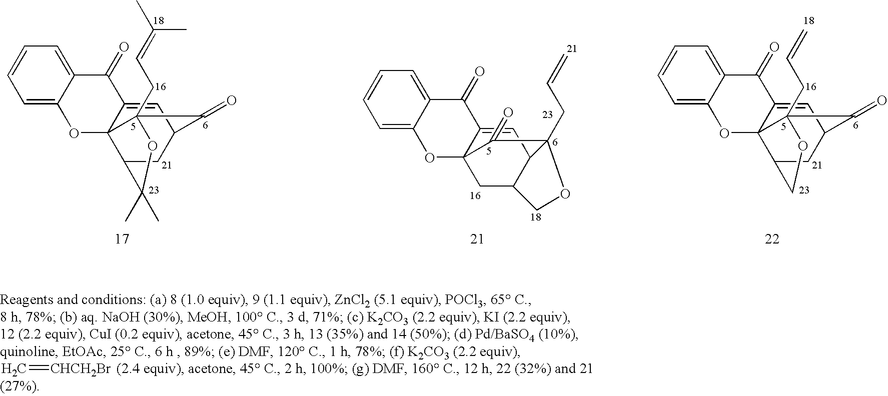

ZnCl₂-induced condensation of o-anisic acid (8) with pyrogallol (9) in POCl₃ produces benzophenone adduct 10 that undergoes a base-induced cyclization to form xanthone 11. Conversion of 11 to bis(dimethylallyloxy)xanthone 15 can be accomplished by a two steps procedure that involves propargylation of the C5 and C6 phenols with 2-chloro-2-methyl butyne (12) to form 14 (see, (a) T. R. Pettus and C. Hoarau, Synlett., 2003, 127-137 and (b) R. Perrin, F. Muyard, F. Bévalot, F. Tillequin and J. Vaquette, J. Nat. Prod., 2000, 63, 245-247) followed by Lindlar reduction of the pendant alkynes (J. Hlubucek, E. Ritchie and W. C. Taylor, Aust. J. Chem., 1971, 24, 2355-2363). The main side-product of this sequence is alkene 13 formed by concomitant reaction of the C5 phenol at the vinyl organometallic intermediate. This compound can be easily separable from desired product 15 via a simple chromatography on silica gel. Heating of 15 in DMF (120° C., 1 h) gives the caged motif 17 via a Claisen-rearranged intermediate 16. In a similar manner compound 11 is allylated with allyl bromide to afford adduct 18. This compound can undergo the Claisen/Diels-Alder reaction at elevated temperatures and prolonged heating (DMF, 160° C., 16 h). In this case the regular caged structure 22 can be formed together with the neo isomer 21.

In one embodiment, the conversion of 15 to 17 can be carried out in a polar solvent, such as water, alcohol or a combination thereof. In one embodiment, the polar solvent is selected from methanol, ethanol, butanol, DMF, THF, CH₃CN, DMSO, water or a combination thereof. In one embodiment, MeOH:H₂O is used as solvent. In one embodiment, the conversion is carried with heating at 100° C. for 0.5 h.

### D. Formulation of Pharmaceutical Compositions

The pharmaceutical compositions provided herein contain therapeutically effective amounts of one or more of compounds provided herein that are useful in the prevention, treatment, or amelioration of one or more of the symptoms of conditions associated with uncontrolled cell growth and spread of abnormal cells, including, but not limited to, cancer.

The compositions contain one or more compounds provided herein.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more diseases associated with uncontrolled cell growth and spread of abnormal cells, including, but not limited to, cancers.

In one embodiment, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more diseases associated with uncontrolled cell growth and spread of abnormal cells, including, but not limited to, cancers.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases associated with uncontrolled cell growth and spread of abnormal cells, including, but not limited to, cancers. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylacetamide (DMA) or dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. The pharmaceutically therapeutically active compounds and derivatives thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in certain embodiments, about 0.1-85% or about 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases associated with uncontrolled cell growth and spread of abnormal cells, including, but not limited to, cancers. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are, in certain embodiments, anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

1. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 gm.

Tablets and capsules for rectal administration are manufactured using the same pharmaceutically acceptable substance and by the same methods as for formulations for oral administration.

6. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions.

Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

7. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

8. Articles of Manufacture

The compounds or pharmaceutically acceptable derivatives can be packaged as articles of manufacture containing packaging material, a compound or pharmaceutically acceptable derivative thereof provided herein, which is used for treatment, prevention or amelioration of one or more symptoms associated with uncontrolled cell growth and spread of abnormal cells, including, but not limited to, cancers, and a label that indicates that the compound or pharmaceutically acceptable derivative thereof is used for treatment, prevention or amelioration of one or more symptoms associated with uncontrolled cell growth and spread of abnormal cells, including, but not limited to, cancers.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated.

### E. Evaluation of the Activity of the Compounds

The ability of the compounds provided herein to inhibit cancer cell growth can be tested by any method known to one of skill in the art, such as using a 3H-thymidine incorporation assay, trypan blue exclusion assay, WST assay that measures metabolic activity of cells alive.

### F. Methods of Use of the Compounds and Compositions

The compounds provided herein are activators of caspases and inducers of apoptosis. Thus, the compounds are useful in a variety of diseases in which there is uncontrolled cell growth and spread of abnormal cells, such as cancers. Examples of cancers include, but are not limited to, non-small cell lung cancer, small cell lung cancer, head and neck squamous cancers, colorectal cancer, prostate cancer, and breast cancer, acute lymphocytic leukemia, adult acute myeloid leukemia, adult non-Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer, and small-cell lung cancer. Childhood cancers amenable to treatment by the methods and with the compositions provided herein include, but are not limited to, brain stem glioma, cerebellar astrocytoma, cerebral astrocytoma, ependymoma, Ewing's sarcoma and family of tumors, germ cell tumor, Hodgkin's disease, ALL, AML, liver cancer, medulloblastoma, neuroblastoma, non-Hodgkin's lymphoma, osteosarcoma, malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, Wilms' tumor, and other childhood kidney tumors.

The methods and compositions provided can also be used to treat cancers that originated from or have metastasized to the bone, brain, breast, digestive and gastrointestinal systems, endocrine system, blood, lung, respiratory system, thorax, musculoskeletal system, and skin.

In certain embodiments, the compounds are activators of caspases and inducers of apoptosis in drug resistant cancer cells, such as breast and prostate cancer cells. In certain embodiments, the compounds are useful for the treatment of a drug resistant cancer.

### G. Combination Therapy

The compounds provided herein may be administered as the sole active ingredient or in combination with other active ingredients. Other active ingredients that may be used in combination with the compounds provided herein include but are not limited to, compounds known to treat conditions associated with uncontrolled cell growth and spread of abnormal cells. In one embodiment, the second active agent used in combination with a compound provided herein is effective in treatment, prevention or amelioration of cancers.

Other active ingredients that may be used in combination with the compounds provided herein include but are not limited to, anti-angiogenesis agents, anti-tumor agents, other cancer treatments and autoimmune agents. Such compounds include, in general, but are not limited to, alkylating agents, toxins, antiproliferative agents and tubulin binding agents. Classes of cytotoxic agents for use herein include, for example, the anthracycline family of drugs, the vinca drugs, the mitomycins, the bleomycins, the cytotoxic nucleosides, the pteridine family of drugs, diynenes, the maytansinoids, the epothilones, the taxanes and the podophyllotoxins. In certain embodiments, the compounds provided herein can be administered in combination with anticancer agents including, but not limited to, alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; RNA/DNA antimetabolites such as 5-azacytidine, 5-fluorouracil and methotrexate; DNA antimetabolites such as 5-fluoro-2′-deoxyuridine, ara-C, hydroxyurea and thioguanine; antibodies such as Herceptin and Rituxan. Other known anti-cancer agents which can be used for combination therapy include melphalan, chlorambucil, cyclophosamide, ifosfamide9 vincristine, mitoguazone, epirubicin, aclarubicin, bleomycin, mitoxantrone, elliptinium, fludarabine9 octreotide, retinoic acid, tamoxifen and alanosine.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

O-anisic acid (8), pyrogallol (9) and 2-chloro-2-methyl butyne (12) were purchased from Aldrich. Gambogic acid (7) was purchased from Gaia Chemical Corporation (CT, USA. All reagents were obtained (Aldrich, Acros) at highest commercial quality and used without further purification except where noted. Air- and moisture-sensitive liquids and solutions were transferred via syringe or stainless steel cannula. Organic solutions were concentrated by rotary evaporation below 45° C. at approximately 20 mmHg. All non-aqueous reactions were carried out under anhydrous conditions, i.e. using flame-dried glassware, under an argon atmosphere and in dry, freshly distilled solvents, unless otherwise noted. Dimethylformamide (DMF) and quinoline were distilled from calcium hydride under reduced pressure (20 mmHg) and stored over 4 Å molecular sieves until needed. Yields refer to chromatographically and spectroscopically (1H NMR, 13C NMR) homogeneous materials, unless otherwise stated. Reactions were monitored by thin-layer chromatography (TLC) carried out on 0.25 mm E. Merck silica gel plates (60F-254) and visualized under UV light and/or developed by dipping in solutions of 10% ethanolic phosphomolybdic acid (PMA) or p-anisaldehyde and applying heat. E. Merck silica gel (60, particle size 0.040-0.063 mm) was used for flash chromatography. Preparative thin-layer chromatography separations were carried out on 0.25 or 0.50 mm E. Merck silica gel plates (60F-254). NMR spectra were recorded on Varian Mercury 400 and/or Unity 500 MHz instruments and calibrated using the residual undeuterated solvent as an internal reference.

The following abbreviations were used to explain the multiplicities: s=singlet, d=doublet, t=triplet, q=quartet, m=multiplet, b=broad. IR spectra were recorded on a Nicolet 320 Avatar FT-IR spectrometer and values are reported in cm-1 units. High resolution mass spectra (HRMS) were recorded on a VG 7070 HS mass spectrometer under chemical ionization (CI) conditions or on a VG ZAB-ZSE mass spectrometer under fast atom bombardment (FAB) conditions.

### Example 1

### Synthesis of the Compounds

### I. (2-methoxyphenyl)(2,3,4-trihydroxyphenyl)methanone (10)

To a 250 ml round-bottomed flask containing flame-dried under vacuum ZnCl₂ (7.00 g, 51.5 mmol) was added o-anisic acid (8) (1.52 g, 10 mmol) and pyrogallol (9) (1.39 g, 11 mmol) followed by POCl₃ (15.0 mL). The reaction vessel was then equipped with a reflux condenser and stirred under argon at 65° C. for 8 hours. The red colored reaction mixture was then cooled to 25° C. and poured into a beaker of about 500 g of ice. The mixture was extracted with ethyl ether (3×100 mL), and the combined organic layers were dried over MgSO₄, filtered, and concentrated. The crude material was purified through column chromatography (30% Et₂O-hexane) to yield benzophenone 10 (2.04 g, 65%).

10: yellow solid; R_{f}=0.65 (90% Et₂O-hexane); ¹H NMR (400 MHz, CDCl₃) δ 7.45 (ddd, J=8.4, 7.2, 1.6 Hz, 1H), 7.26 (dd, J=7.2, 2 Hz, 1H), 7.04-6.98 (m, 2H), 6.87 (d, J=9.2 Hz, 1H), 6.42 (d, J=9.2 Hz, 1H), 3.77 (s, 3H); ¹³C NMR (100 MHz, CDCl₃) δ 200.5, 156.2, 151, 150.1, 131.5, 130.8, 128.7, 127.4, 126.5, 120.2, 114.2, 111.3, 107.1, 55.7; HRMS calc. for C₁₄H₁₂O₅ (M⁺H⁺) 261.0763, found 261.0741.

### II. 3,4-dihydroxy-9H-xanthen-9-one (11)

To a solution of benzophenone 10 (1.6 g, 6.15 mmol) in methanol (20 mL) was added a solution of aqueous NaOH (30% ww, 20 mL) and water (10 mL). The green colored reaction mixture was then refluxed at 100° C. for 3 days. The red colored reaction mixture was cooled to 25° C. and acidified with aqueous HCl (10%, 600 mL). The reaction mixture was partitioned between water and ethyl ether (50 mL). The aqueous layer was then back extracted with ethyl ether (2×50 mL). The combined organic layers were dried over MgSO₄, filtered, and concentrated. The crude material was purified through column chromatography (20-40% Et₂O in hexanes) to yield xanthone 11 (0.99 g, 71%). 11: yellow solid; R_{f}=0.4 (90% Et₂O-hexane); ¹H NMR (400 MHz, DMSO) δ 8.14 (dd, J=8.0, 1.6 Hz, 1H), 7.82-7.78 (m, 1H), 7.62 (d, J=7.6 Hz, 1H), 7.54 (d, J=8.8 Hz, 1H), 7.43-7.39 (m, 1H), 7.0 (d, J=8.8 Hz, 1H); ¹³C NMR (100 MHz, DMSO) δ 175.1, 155.3, 151.4, 146.2, 134.6, 132.5, 125.7, 123.8, 120.7, 117.9, 116.4, 114.6, 113.2; HRMS calc. for C₁₃H₈O₄ (M⁺H⁺) 229.0501, found 229.0509.

### III. 3,4-bis(2-methylbut-3-yn-2-yloxy)-9H-xanthen-9-one (14)

To a round-bottomed flask containing xanthone 11 (500 mg, 2.19 mmol), KI (800 mg, 4.82 mmol), K₂CO₃ (666.2 mg, 4.82 mmol), and CuI (42 mg, 0.22 mmol) was added dry acetone (20 mL). The reaction vessel was then equipped with a reflux condenser, and the reaction was heated at 45° C. under argon. After 20 minutes, 2-chloro-2-methylbut-3-yne (12) (0.55 ml, 4.82 mmol) was added, and the reaction was heated for two more hours. The reaction was then cooled to 25° C. and acidified with 10% HCl solution. The reaction mixture was partitioned between ethyl ether (30 mL) and water. The aqueous layer was back-extracted (2×30 mL), and the combined ethyl ether layers was dried over MgSO₄, filtered, and concentrated. The crude material was purified through a column chromatography (5-20% Et₂O in hexane) to yield compound 14 (394.8 mg, 50%).

14: yellow solid; R_{f}=0.45 (50% Et₂O-hexane); ¹H NMR (400 MHz, CDCl₃) δ 8.32 (dd, J=8.0, 1.2 Hz, 1H), 8.06 (d, J=9.2 Hz, 1H), 7.71-7.67 (m, 1H), 7.65 (d, J=9.2 Hz, 1H), 7.51 (d, J=8.4 Hz, 1H), 7.36 (dd, J=8.0, 7.2 Hz, 2.66 (s, 1H), 2.29 (s, 1H), 1.83 (s, 6H), 1.76 (s, 6H); ¹³C NMR (100 MHz, CDCl₃) δ 176.5, 155.8, 155.7, 152.2, 134.2, 126.4, 123.7, 121.7, 117.9, 115.8, 74.8, 73.6, 30.6, 29.7; HRMS calc. for C₂₃H₂₀O₄ (M⁺H⁺) 361.1440, found 361.1464.

### IV. 3,4-bis(2-methylbut-3-en-2-yloxy)-9H-xanthen-9-one (15)

To a solution of xanthone 14 (100 mg, 0.28 mmol) in EtOAc (4 mL) was added 10% Pd/BaSO₄ (10 mg) and quinoline (0.66 mL, 0.56 mmol). The reaction mixture was degassed using argon and stirred under an atmosphere of hydrogen for 6 hours. During that time, an additional amount of 10% Pd/BaSO₄ (10 mg) was added to accelerate the reaction. The reaction mixture was filtered through a plug of silica gel, and the residue concentrated and purified through a column chromatography (3-10% Et₂O-hexane) to yield 15 (90.1 mg, 75%).

15: yellow solid; R_{f}=0.66 (70% Et₂O-hexane); ¹H NMR (400 MHz, CDCl₃) δ 8.26 (dd, J=6.0, 1.6 Hz, 1H), 7.90 (d, J=8.8, 0.8 Hz, 1H), 7.65-7.61 (m, 1H), 7.46-7.43 (m, 1H), 7.31-7.27 (m, 1H), 7.09 (dd, J=9.2, 2.0 Hz, 1H), 6.29-6.10 (m, 2H), 5.18-5.11 (m, 3H), 4.99-4.96 (m, 1H), 1.54 (d, J=2 Hz, 6H), 1.52 (d, J=2.4 Hz, 6H); ¹³C NMR (100 MHz, CDCl₃) δ 176.2, 156.5, 155.5, 152.1, 143.0, 135.4, 134.0, 126.2, 123.8, 123.5, 121.2, 120.8, 120.7, 117.7, 117.6, 116.9, 116.4, 113.8, 112.8, 83.3, 81.9, 27.1, 26.9; HRMS calc. for C₂₃H₂₄O₄ (M⁺H⁺) 365.1753, found 365.1740.

### V. Caged Xanthone 17

A solution of 15 (55 mg, 0.15 mmol) in DMF (2.0 mL) was heated at 120° C. for 1 hour. The yellow reaction mixture was cooled 25° C. and the mixture purified by column chromatography (15-20% Et₂O-hexane) to yield the caged xanthone 17 (42.5 mg, 78%).

17: white solid; R_{f}=0.55 (70% Et₂O-hexane); ¹H NMR (400 MHz, CDCl₃) δ 7.93 (dd, J=8.0, 1.6 Hz, 1H), 7.53-7.49 (m, 1H), 7.42 (d, J=6.8 Hz, 1H), 7.07-7.03 (m, 2H), 4.42-4.38 (m, 1H), 3.49 (dd, J=6.8, 4.8 Hz, 1H), 2.67-2.61 (m, 2H), 2.45 (d, J=9.6 Hz, 1H), 2.33 (dd, J=13.6, 4.8 Hz, 1H), 1.72 (s, 3H), 1.30 (s, 6H), 0.89 (s, 3H); ¹³C NMR (100 MHz, CDCl₃) δ 202.7, 176.2, 159.4, 136.0, 134.7, 134.6, 133.5, 126.8, 121.7, 118.9, 118.8, 117.9, 90.2, 84.5, 83.5, 48.8, 46.8, 30.4, 29.8, 29.2, 25.4, 25.2, 16.8; HRMS calc. for C₂₃H₂₄O₄ (M⁺H⁺) 365.1753, found 365.1765.

### VI. 3,4-bis(allyloxy)-9H-xanthen-9-one (18)

To a flask containing xanthone 11 (500 mg, 2.19 mmol) and K₂CO₃ (666.2 mg, 4.82 mmol) was added dry acetone (20 mL), followed by allylbromide (0.42 mL, 4.82 mmol). The reaction vessel was then equipped with a reflux condenser, and the reaction was heated at 45° C. under argon for two hours. The mixture was then cooled to room temperature and acidified with 10% aqueous HCl solution. The reaction mixture was partitioned between ethyl ether (20 mL) and water (20 mL). The aqueous layer was back-extracted (2×20 mL), and the combined ethyl ether layers were dried over MgSO₄, filtered, and concentrated. The crude material was purified through a column chromatography (5-20% Et₂O-hexane) to yield allylated xanthone 18 (675.2 mg, 100%).

18: white solid; R_{f}=0.45 (50% Et₂O-hexane); ¹H NMR (400 MHz, CDCl₃) δ 8.3 (dd, J=8.0, 1.6 Hz, 1H), 8.04 (d, J=8.8 Hz, 1H), 7.69 (dt, J=8.4, 1.2 Hz, 1H), 7.54 (d, J=8.4 Hz, 1H), 7.35 (dd, J=8.0, 7.2 Hz), 6.97 (d, J=8.8 Hz), 6.23-6.03 (m, 2H), 5.49-5.32 (m, 3H), 5.38 (dd, J=10.4, 0.8 Hz, 1H), 4.71 (dd, J=6.4, 5.2 Hz); ¹³C NMR (100 MHz, CDCl₃) δ 176.2, 156.5, 155.9, 150.6, 134.3, 133.6, 132.1, 126.4, 123.7, 122.1, 121.4, 118.3, 118.1, 117.9, 116.6, 109.8, 74.7, 69.8; HRMS calc. for C₁₉H₁₆O₄ (M+H⁺) 309.1127, found 309.1134.

### VII. Caged Xanthones 21 and 22

A solution of 18 (46.2 mg, 0.15 mmol) in DMF (2.0 mL) was heated at 160° C. for 12 h. The yellow reaction mixture was then cooled to 25° C. and the residue was column chromatographed (10-30% Et₂O-hexane) to yield a mixture of caged xanthones 21 and 22.

21: (12.4 mg, 27%), white solid; R_{f}=0.55 (70% Et₂O-hexane); ¹H NMR (300 MHz, CDCl₃) δ 7.95 (d, J=7.8 Hz, 1H), 7.59-7.54 (m, 1H), 7.34 (d, J=7.2 Hz, 1H), 7.11-6.98 (m, 2H), 5.29-5.15 (m, 1H), 4.68 (d, J=9.9 Hz, 1H), 4.56-4.50 (m, 2H), 3.91 (d, J=7.5 Hz, 1H), 3.54-3.44 (m, 1H), 2.61 (m, 2H), 2.23 (m, 3H); HRMS calc. for C₁₉H₁₆O₄ (M+H⁺) 309.1127, found 309.1132. 22: (14.7 mg, 32%), white solid; R_{f}=0.54 (70%, Et₂O-hexane); ¹H NMR (400 MHz, CDCl₃) δ 7.92 (dd, J=8.0, 2.0 Hz, 1H), 7.58-7.53 (m, 1H), 7.30 (d, J=6.8 Hz, 1H), 7.18 (d, J=8.4 Hz, 1H), 7.09-7.05 (m, 1H), 5.65-5.54 (m, 1H), 5.14-5.09 (m, 2H), 4.08 (dd, J=8.4, 3.6 Hz, 1H), 3.94 (m, 1H), 3.47 (dd, J=6.4, 4.4 Hz, 1H), 2.63 (dd, J=14, 6 Hz, 1H), 2.57-2.48 (m, 1H), 2.28 (dd, J=14, 7.6 Hz, 1H), 2.21 (d, J=12.8, 5.6 Hz, 2H); ¹³C NMR (100 MHz, CDCl₃) δ 198.1, 175.4, 160.2, 136.6, 136.5, 134.5, 134.3, 134.2, 131.6, 131.4, 127.2, 126.9, 122.3, 121.9, 119.6, 119.1, 118.5, 118.2, 83.7, 76.2, 75.9, 45.7, 45.4; HRMS calc. for C₁₉H₁₆O₄ (M+H⁺) 309.1127, found 309.1133.

### Example 2

### Biological Activity

### I. ³H-Thymidine Incorporation Assay.

The ability of the compounds to inhibit cancer cell growth was evaluated in promyelocytic leukemia cell line, HL-60, using a ³H-thymidine incorporation assay. Cells were plated in a 96-well plate at 10-20×10³ cells/well in RPMI supplemented with 10% fetal bovine serum, 2 mM glutamine, 1% penicillin/streptomycin (complete medium). The caged Garcinia xanthones were added to the cells at increasing concentrations and 0.1% DMSO was added to control cells. Cells were incubated for 48 h and then pulsed with ³H-thymidine for 6 h. Incorporation of ³H-thymidine was determined in a scintillation counter (Beckman Coulter Inc., Fullerton, Calif.) after cells were washed and deposited onto glass microfiber filters using a cell harvester M-24 (Brandel, Gaithersburg, Md.). Table 1 provides IC₅₀ values for the natural products gambogic acid and gambogin, forbesione and deoxymorellin.

The compounds were also evaluated in HL-60/ADR cells, a multidrug resistant clone obtained by transfection of HL-60 cells with mdr-1. (See, A. Batova, L. E. Shao, M. B. Diccianni, A. L. Yu, T. Tanaka, A. Rephaeli, A. Nudelman and J. Yu, Blood, 2002, 100, 3319-24). The results of parallel experiments indicated that HL-60/ADR had similar sensitivity to the anti-proliferative effects of the caged Garcinia xanthones as the parental HL-60 cell line. Without being bound to any particular theory, it is believed that the caged Garcinia xanthones are not subject to the mechanism of chemo-resistance resulting from the expression of mdr, characteristic of many relapsed cancers. (See, M. M. Gottesman and I. Pastan, Annu. Rev. Biochem., 1993, 62, 385-427; S. Simon, D. Roy and M. Schindler, PNAS, 1994, 91, 1128-1132 and A. K. Larsen, A. E. Escargeuil and A. Skladanowski, Pharmacol. Ther., 2000, 85, 217-229).

TABLE 1
Inhibition of cell proliferation by caged Garcinia xanthones in
adriamycin sensitive and resistant promyelocytic leukemia cells

| Compound | HL-60 | HL-60/ADR |
|---|---|---|
| Forbesione | 2.2 | 2.0 |
| desoxymorellin | 1.0 | 1.1 |
| gambogic acid | 0.3 | 0.5 |
| gambogin | 0.8 | 1.1 |
| 17 | 1.5 | 1.4 |
| 21 | <10% inhibition ar 2 μm | <10% inhibition ar 2 μm |
| 22 | <10% inhibition ar 2 μm | <10% inhibition ar 2 μm |

Analogue 17 showed similar activity to that of the natural products (IC₅₀=1.5 μM), while the related structures 21 and 22 induced less than 10% growth inhibition at the highest concentrations tested (2.0 μM).

### II. Trypan-Blue Exclusion Assay.

Compounds such as desoxymorellin, gambogic acid, gambogin and 17 were further evaluated in a panel of solid and non-solid tumor cell lines. CEM cells were plated in a 24-well plate in complete media at 50×10³ cells/well. Cells were treated with gambogin at concentrations of 0.25, 0.5, 1.0 μM or with 0.1% DMSO (control cells). Cells were incubated for 4 days and then the number of viable cells was determined after the addition of trypan-blue dye by counting the cells which exclude trypan-blue in a hemocytometer. Data for inhibition of cell proliferation is provided in.

TABLE 2
Inhibition of cell proliferation by caged Garcinia
xanthones in solid and non-solid tumor cells

| | | Compound | | | |
|---|---|---|---|---|---|
| | | desoxymorellin | gambogic acid | gambogin | 17 |
| cell line | tissue type | IC₅₀ (μM) | | | |
| A549 | lung | 2.1 | 1.8 | 1.8 | >4 |
| HT29 | colon | 1.2 | 0.7 | 1.0 | 3.1 |
| MCF-7 | breast | 0.9 | 0.4 | 1.1 | ND* |
| M21 | melanoma | 1.6 | 1.2 | 1.0 | 2.2 |
| PC3 | prostate | 1.2 | 0.4 | 1.1 | ND* |
| CEM | leukemia | ND* | 0.15 | 0.35 | 0.3 |

*ND: Not determined

The T-cell acute lymphoblastic leukemia cell line (CEM) was the most sensitive among all the cell lines tested. The IC₅₀ values recorded in these cells were in the submicromolar range (0.15-0.35 μM). The solid tumor cell lines were slightly less sensitive than CEM cells with IC₅₀ values of the compounds ranging from 0.4 to 3.1 μM, with the exception of compound 17 in A549 cells (IC₅₀>4 μM).

### III. WST Assay.

Cell viability was determined using the WST assay in HUVE (human umbilical vein endothelial) cells that measures metabolic activity of cells alive. Compound 17 was dissolved in DMSO and further diluted with Endothelial Cell Growth Medium (PromoCell, Heidelberg, Germany) to obtain a final concentration as indicated. HUVE cells (PromoCell, Heidelberg, Germany) were seeded into each well of a 96-well cell culture plate at 7000 cells per well and incubated at 37° C. for 24 h with the indicated concentrations of each compound. The final volume was 100 μL per well. Control samples were incubated with the solvent alone. Each experiment was repeated in triplicate. Afterwards the WST-1 reagent was added to the cells at 10 μL per well and the cells further incubated at 37° C. for additional 3 h. Then the cell culture plate was agitated thoroughly for 1 minute on a shaker at 200 U/min. The absorbance of each sample was measured using a microplate reader at 440 nm. The reference wavelength was 690 nm.

Compound 17 was found to be cytotoxic with an IC₅₀ value of 1.38 μM.

### IV. Apoptosis Assay.

The compounds were dissolved in DMSO and further diluted with Endothelial Cell Growth Medium (PromoCell, Heidelberg, Germany) to obtain final concentrations as indicated. HUVE cells were seeded into each well of a 96-well cell culture plate at 10000 cells per well and incubated at 37° C. for 10 h with the indicated concentrations of each compound. The final volume was 100 μL per well. Control samples were incubated with the solvent alone. Each sample was repeated three times. The proapoptotic effect was detected by using the Cell Death Detection ELISA^{PLUS} kit (Roche Diagnostics GmbH, Mannheim, Germany) according to the manufactor's instructions. The kit constitutes a photometric enzyme-immunoassay for the qualitative and quantitative in vitro determination of cytoplasmic histone-associated-DNA-fragments (mono- and oligo-nucleosomes) after induced cell death. Due to the working procedure the kind of cell death (apoptosis or necrosis) can be determined. The absorption values A (A₄₀₅ₙₘ-A₄₉₀ₙₘ) measured give a quantitative indication of the induced amount of apoptosis/necrosis. The higher the absorption A, the higher the induction of apoptosis/necrosis at the corresponding concentrations of the compounds.

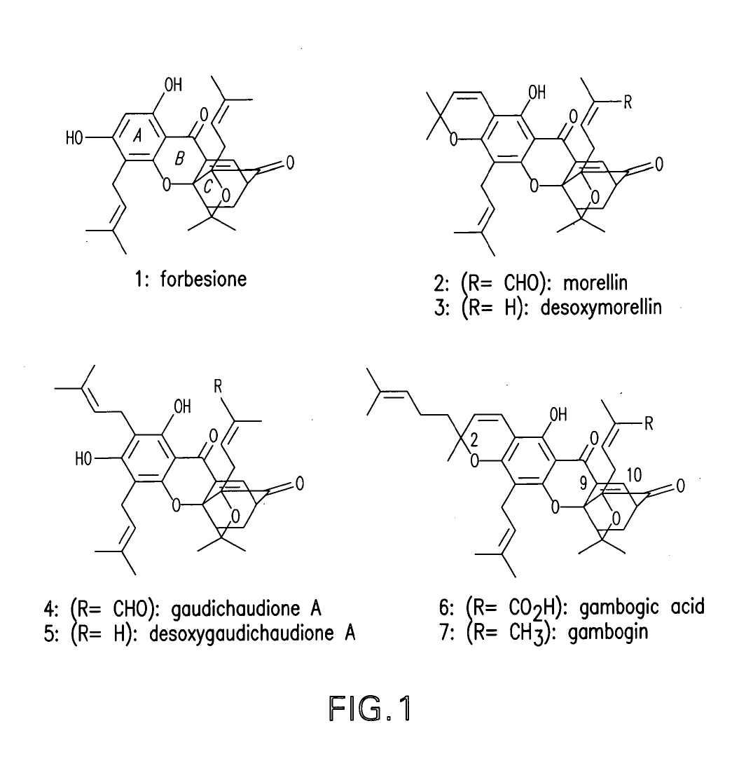

The majority (>90%) of HUVE cells treated with 17 underwent rapid apoptosis after 10 h in a dose-dependent manner (FIG. 1). Cell necrosis was not detected at concentrations lower than 1.5 μM and was only observed in a small subset of cells (ca 10%) at much higher concentrations (>3 μM), indicating that apoptosis is the predominant mechanism of cell death.

### V. Animal Studies

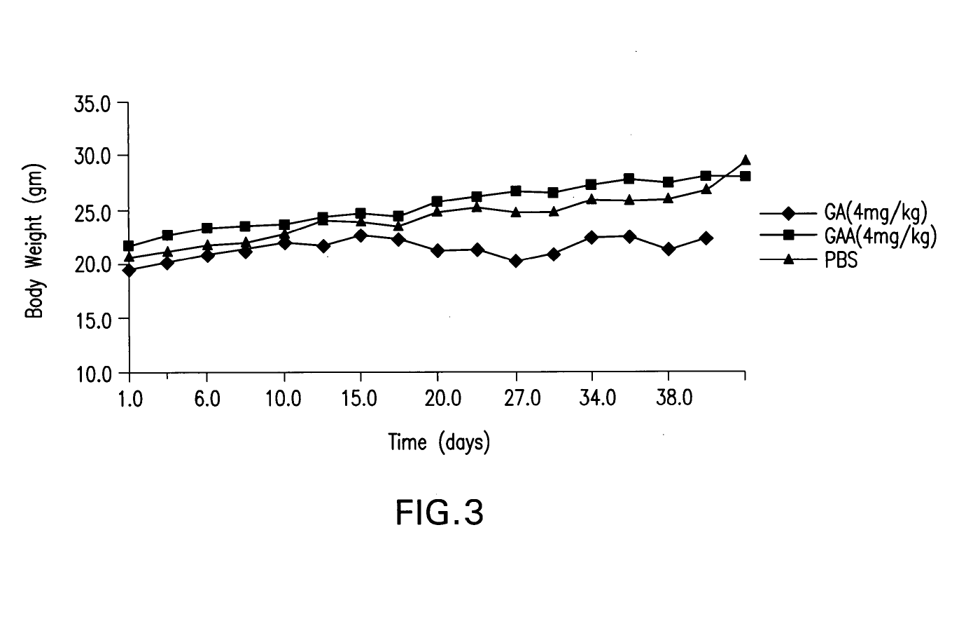

SCID mice were injected with primary human leukemia cells. Seven days following injection of leukemia cells, mice were given IV injections of gambogic acid, gambogic acid analog (17), or PBS (control). Each treatment group contained 3 mice which were monitored for body weight upon each injection. The results are shown in FIG. 3. While gambogic acid showed some toxicity, there was no significant toxicity for gambogic acid analog (17) when compared to PBS control.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

1. A compound of formula I or II: 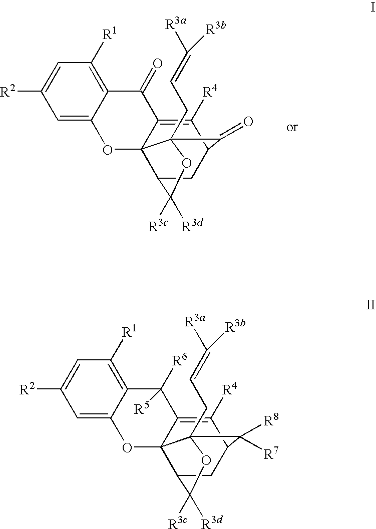
or a pharmaceutically acceptable derivative thereof, wherein
R¹ and R² are each independently hydrogen, alkyl, or —OR^{a};
R^{a} is hydrogen, alkyl, alkenyl, alkynyl, cycloalkyl or aryl;
R^{3a}, R^{3b}, R^{3c} and R^{3d} are each independently hydrogen, alkyl, alkenyl or alkynyl;
R⁴ is hydrogen, halo, pseudohalo, hydroxy, oxo, alkyl, alkenyl, alkynyl, cycloalkyl, aryl, —OR^{a}, —S(O)ₙR^{b}, —OC(O)R^{b}, —OC(O)OR^{b}, or —NR^{c}R^{d}, —NR^{c}COR^{d} or —NR^{c}SO₂R^{d};
R^{a}, R^{b}, R^{c} and R^{d} are each independently hydrogen, alkyl, alkenyl, alkynyl, aryl, alkylaryl, heterocycle, aralkyl, aralkoxy, cycloalkyl, cycloalkenyl or cycloalkynyl;
n is 0-2;
R⁵ and R⁷ are each independently hydrogen, alkyl, alkenyl or alkynyl; and
R⁶ and R⁸ are each independently hydrogen, alkyl, alkenyl, alkynyl or —OR^{a},
where R¹, R², R^{3a}, R^{3b}, R^{3c}, R^{3d}, R⁴, R⁵, R⁶, R⁷, R^{a}, R^{b}, R^{c} and R^{d} are optionally substituted with 1, 2, 3 or 4 substituents, each independently selected from Q¹, where Q¹ is halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl, alkynyl, heteroalkyl, cycloalkyl, cycloalkylalkyl, heterocyclyl, heterocyclylalkyl, aryl, heteroaryl, aralkyl, aralkenyl, aralkynyl and heteroarylalkyl.

2. The compound of claim 1, wherein Q¹ is alkyl, halo, hydroxy or haloalkyl.

3. The compound of claim 1, wherein R¹ is hydrogen.

4. The compound of claim 1,
wherein R² is hydrogen.

5. The compound of claim 1, wherein R^{3a} is hydrogen or lower alkyl.

6. The compound of claim 1, wherein R^{3a} is hydrogen or methyl.

7. The compound of claim 1, wherein R^{3b} is hydrogen or lower alkyl.

8. The compound of claim 1, wherein R^{3b} is hydrogen or methyl.

9. The compound of claim 1, wherein R^{3c} is hydrogen or lower alkyl.

10. The compound of claim 1, wherein R^{3c} is hydrogen or methyl.

11. The compound of claim 1, wherein R^{3d} is hydrogen or lower alkyl.

12. The compound of claim 1, wherein R^{3d} is hydrogen or methyl.

13. The compound of claim 1, wherein R^{3a}, R^{3b}, R^{3c} and R^{3d} are each methyl.

14. The compound of claim 1, wherein R⁴ is hydrogen, alkyl or —OR^{a}.

15. The compound of claim 1, wherein R⁴ is hydrogen.

16. The compound of claim 1, wherein R⁵ and R⁷ are each hydrogen.

17. The compound of claim 1, wherein R⁶ and R⁸ are each hydrogen.

18. The compound of claim 1, wherein the compound has formula

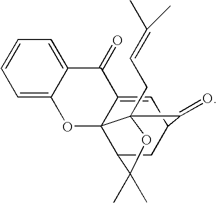

19. A pharmaceutical composition comprising the compound of claim 1, and a pharmaceutically acceptable carrier.

20. A method for treating or ameliorating a disease associated with uncontrolled cell growth comprising administering a compound of claim 1.

21. The method of claim 20, wherein the disease is non-small cell lung cancer, small cell lung cancer, head and neck squamous cancers, colorectal cancer, prostate cancer, breast cancer, acute lymphocytic leukemia, adult acute myeloid leukemia, adult non-Hodgkin's lymphoma, brain tumor, cervical cancer, childhood cancer, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma or skin cancer.

23. The method of claim 21 further comprising administering a second agent selected from an alkylating agent, cytotoxic agent, antiproliferative agent, tubulin binding agent and a combination thereof.

24. A process for synthesis of the compound of claim 18 comprising reacting o-anisic acid and pyrogallol to obtain a benzophenone adduct 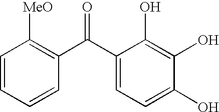
(2-methoxyphenyl)(2,3,4-trihydroxyphenyl)methanone.

24. The process of claim 23 further comprising cyclization of (2-methoxyphenyl)(2,3,4-trihydroxyphenyl)methanone to obtain 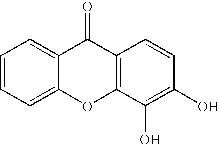
3,4-dihydroxy-9H-xanthen-9-one.

25. The process of claim 24 further comprising reacting 3,4-dihydroxy-9H-xanthen-9-one with 2-chloro-2-methylbut-3-yne followed by Lindlar reduction to obtain 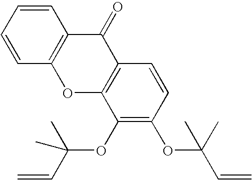
3,4-bis(2-methylbut-3-en-2-yloxy)-9H-xanthen-9-one.

26. The process of claim 25 further comprising heating 3,4-bis(2-methylbut-3-en-2-yloxy)-9H-xanthen-9-one in a polar solvent to obtain

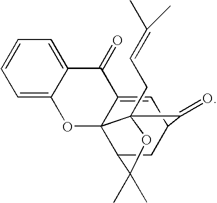

27. The process of claim 26, wherein the polar solvent is methanol, ethanol, butanol, DMF, THF, CH₃CN, DMSO, water or a combination thereof.

28. The process of claim 27, wherein the polar solvent is dimethyl formamide or CH₃OH/H₂O.
